(19) United States
(12) Patent Application Publication
(10) Pub. No.: US 2012/0107376 A1
(43) Pub. Date: May 3, 2012

(54) METHOD FOR IMPROVING BIOAVAILABILITY OF LATANOPROST

(75) Inventors: Timo Reunamaki, Tampere (FI); Paula Tervo, Tampere (FI); Jukka Lokkila, Ruutana (FI); Pertti Pellinen, Lempaala (FI); Paivi Alajuuma, Valkeakoski (FI); Olli Oksala, Tampere (FI)

(73) Assignee: Santen Pharmaceutical Co. Ltd

(21) Appl. No.: 13/381,631

(22) PCT Filed: Jun. 23, 2010
(86) PCT No.: PCT/JP2010/004179

§ 371 Date: Dec. 29, 2011

(30) Foreign Application Priority Data
Jun. 30, 2009 (EP) 09397519.1

(51) Int. Cl. A61K 31/216; A61P 27/06; A61K 9/00

(52) U.S. Cl. 424/400; 514/530

(57) ABSTRACT

The present invention relates to a method for improving ocular bioavailability of latanoprost by adding an organic amine to an aqueous eye drop composition containing latanoprost. The invention further relates to an aqueous eye drop composition in which a better ocular bioavailability of latanoprost is achieved by adding an organic amine, and to a method for treating occular hypertension and glaucoma by administering said composition to a subject in need of such treatment.

### TECHNICAL FIELD

The present invention relates to a method for improving ocular bioavailability of latanoprost by adding an organic amine to an aqueous eye drop composition containing latanoprost. The invention further relates to an aqueous eye drop composition in which a better ocular bioavailability/penetration of latanoprost is achieved by adding an organic amine to the aqueous eye drop composition containing latanoprost, and to a method for treating ocular hypertension and glaucoma by administering said composition to a subject in need of such treatment. The composition is well tolerated.

### BACKGROUND ART

Like other prostaglandin derivatives, such as isopropyl unoprostone, tafluprost or travoprost, latanoprost is a highly lipophilic medicament. The concentration of latanoprost used for the treatment of glaucoma is very low, approximately 0.005% (w/v). However, although latanoprost is effective in low amounts, there is still a need to reduce that minor amount to its effective minimum to be administered onto the eye surface. On the other hand, when the amount of the active agent in an eye drop composition is lowered, a balanced but sufficient penetration may become critical or impossible to obtain repeatedly and safely.

Adjuvant agents are included in aqueous eye drop composition in order to achieve sufficiently stable and effective eye drops for the desired purposes. Preservatives have been included in aqueous eye drop compositions not only for their antimicrobial effect but also for their stabilising and homogenizing interactions with the other ingredients. However, preservatives are also known as the major etiology of keratoconjuctive disorders, and for safety purposes it is preferred that the concentration of preservatives, such as those of benzalkonium chloride (BAK) type, is as low as possible. Animal studies and in vitro studies have demonstrated that preservatives such as BAK cause harmful effects on several eye tissues, including the tear film, cornea, conjunctiva and trabecular cells (Baudouin C., Detrimental effect of preservatives in eyedrops: implications for the treatment of glaucoma. Acta Ophthalmol 2008; 86:716-726 (Non-Patent Document 1)). Evidence from some rabbit and human studies has also suggested that the presence of BAK may affect the corneal epithelium layer and thus increase the transcorneal penetration of poorly permeable drugs. However, a recent study has shown that the corneal penetration into rabbit aqueous humor is comparable between BAK-preserved and preservative-free tafluprost, which is another member in the family of prostaglandin derivatives (Pellinen P, Lokkila J., Corneal penetration into rabbit aqueous humor is comparable between preserved and preservative-free tafluprost. Ophthalmic Res 2009; 41:118-122(Non-Patent Document 2)). The dual role of preservatives (both antimicrobial and stabilizing/solubilizing effects) has lead to difficulties e.g. in preparing stable, preservative-free unit dose eye drop preparations.

Nonionic surfactants and antioxidants have been added to ophthalmic aqueous prostaglandin derivative solutions e.g. in order to prevent lowering of concentration of said prostaglandin derivatives. The latanoprost composition of WO2004/037267 (Sucampo) (Patent Document 1) comprises polysorbate or polysorbate and EDTA, but substantially no BAK. In EP 1321144 (Santen) (Patent Document 2), nonionic surfactants, especially polysorbates, have been found to prevent a prostaglandin derivative from being adsorbed to resinous container walls. The use of nonionic surfactants has been almost inavoidable in ophthalmic aqueous compositions comprising prostaglandin derivatives, especially if a preservative-free aqueous composition has been desired to be produced.

Organic amines have been used in several ophthalmic compositions, mostly as buffers and pH adjusting agents. Japanese Patent Laying-Open No. 2003-146881 (Toyo) (Patent Document 3) discloses the prevention of precipitation of anti-allergic pemirolast potassium using trometamol as buffering agent. According to Japanese Patent Laying-Open No. 2003-327530 (Sankyo) (Patent Document 4) the preservative effect of a cationic disinfectant such as benzalkonium chloride can be enhanced with trometamol. Incorporating trometamol with a non-ionic surfactant into a suspension-type preparation of an oxazine-picolinic acid has been found to result in suppression of the adhesion of the active ingredient (WO2008/111630 (Santen) (Patent Document 5)). An antiseptic ophthalmic composition free from irritation to eyes can be formulated by including i.a. trometamol in the composition (Japanese Patent Laying-Open No. 2004-002364 (Lion) (Patent Document 6); Japanese Patent Laying-Open No. 2003-300871 (Lion) (Patent Document 7) and Japanese Patent Laying-Open No. 2003-206241 (Teika) (Patent Document 8). According to US2003/0055051 (Wakamoto) (Patent Document 9) an excellent but less stimulative ophthalmic anti-inflammatory effect can be obtained by adding trometamol to an ophthalmic preparation. In Japanese Patent Laying-Open No. 11-302162 (Kissei) (Patent Document 10), a better anti-allergic effect of the active ingredient is obtained by incorporating trometamol. Promotion of intraocular penetration of a phenylacetic acid NSAID-compound is possible from a composition containing trometamol (EP 1808170 (Senju) (Patent Document 11). According to EP 1916002 (Santen) (Patent Document 12), degradation of a thermally unstable medicament, such as latanoprost, can be prevented by adding an organic amine such as trometamol.

However, despite of the above mentioned several references of eye drop formulations and various organic amines, there is no disclosure or suggestion that adding an organic amine to an ophthalmic aqueous preservative-free composition, wherein the active ingredient is highly lipophilic like latanoprost could cause a better transcorneal penetration of the latter and hence improved ocular bioavailability of latanoprost in relation to the reduced administered amount.

### CITATION LIST

### Patent Literature

PTL 1: WO2004/037267
PTL 2: EP 1321144
PTL 3: Japanese Patent Laying-Open No. 2003-146881
PTL 4: Japanese Patent Laying-Open No. 2003-327530
PTL 5: WO2008/111630
PTL 6: Japanese Patent Laying-Open No. 2004-002364
PTL 7: Japanese Patent Laying-Open No. 2003-300871
PTL 8: Japanese Patent Laying-Open No. 2003-206241
PTL 9: US2003/0055051
PTL 10: Japanese Patent Laying-Open No. 11-302162
PTL 11: EP 1808170
PTL 12: EP 1916002

### Non-Patent Literature

NPL 1: Baudouin C., Detrimental effect of preservatives in eyedrops: implications for the treatment of glaucoma. Acta Ophthalmol 2008; 86:716-726
NPL 2: Pellinen P, Lokkila J., Corneal penetration into rabbit aqueous humor is comparable between preserved and preservative-free tafluprost. Ophthalmic Res 2009; 41:118-122

### SUMMARY OF INVENTION

### Technical Problem

The present invention seeks to solve the above problems, and it is therefore an object of the present invention to provide a method for improving ocular bioavailability of preservative-free latanoprost in an aqueous eye drop composition by adding an organic amine to the eye drop composition containing latanoprost.

Another object of the invention is a preservative-free aqueous eye drop composition comprising latanoprost, wherein the ocular bioavailability of latanoprost is improved by adding an organic amine.

A further object of the invention is a method for treating ocular hypertension and glaucoma by administering to a subject in need of such treatment an effective amount of an aqueous preservative-free eye drop composition comprising latanoprost, wherein the ocular bioavailability of latanoprost is improved by adding an organic amine.

### Solution to Problem

The present invention is directed to a method for improving ocular bioavailability of latanoprost from an aqueous eye drop composition by adding an organic amine to the eye drop composition containing latanoprost.

In this regard, it is preferred that the organic amine is an organic amine having a hydroxy group, and the organic amine having a hydroxy group is particularly preferably trometamol.

Further, in these methods according to the present invention, it is also preferred that the aqueous eye drop composition contains substantially no preservatives.

It is also preferred that the aqueous eye drop composition contains viscosity enhancing agents.

The present invention is also directed to an aqueous eye drop composition comprising latanoprost, wherein the ocular bioavailability of latanoprost is improved by adding an organic amine.

In this regard, it is preferred that the organic amine is an organic amine having a hydroxy group, and the organic amine having a hydroxy group is particularly preferably trometamol.

Further, in the aqueous eye drop composition comprising latanoprost according to the present invention, it is also preferred that the composition contains substantially no preservatives.

It is also preferred that the composition contains viscosity enhancing agents.

In the aqueous eye drop composition comprising latanoprost according to the present invention, it is preferred that the composition comprises 0.0025 to 0.005% (w/v) latanoprost, 0.1 to 1% (w/v) trometamol, optionally buffering agents, pH adjusters, tonicity and viscosity enhancing agents conventionally used in ophthalmic solutions, and substantially no preservatives, in a unit dose container or fluid dispenser consisting essentially of polyethylene or in contact with container material consisting essentially of polyethylene.

Moreover, the present invention is also directed to a method for treating ocular hypertension and glaucoma by administering to a subject in need of such treatment an effective amount of an aqueous eye drop composition comprising latanoprost, wherein the ocular bioavailability of latanoprost is improved by adding an organic amine.

### Advantageous Effects of Invention

According to the present invention it has now been found that ocular bioavailability of latanoprost in an aqueous eye drop composition can be improved by adding an organic amine to the aqueous eye drop composition containing latanoprost. This invention enables to provide homogenous and stable, preservative-free aqueous ophthalmic latanoprost solution as a sterile, preferably unit dose type formulation. The aqueous eye drop composition according to the invention is also less irritative to the eye than the currently available commercial product. The aqueous eye drop composition of the present invention also remains stable at room temperature, contrary to the currently available commercial product which requires cold storage (at 2-8° C.).

### BRIEF DESCRIPTION OF DRAWINGS

FIG. 1A illustrates absorption of latanoprost from preservative-free test solution A into aqueous humor of rabbit eye in comparison with Xalatan.

FIG. 1B illustrates absorption of latanoprost from preservative-free test solution B into aqueous humor of rabbit eye in comparison with Xalatan.

FIG. 1C illustrates absorption of latanoprost from preservative-free test solution C into aqueous humor of rabbit eye in comparison with Xalatan.

FIG. 1D illustrates absorption of latanoprost from preservative-free test solution D into aqueous humor of rabbit eye in comparison with Xalatan.

FIG. 2A shows intraocular pressure(IOP) after single topical administration in rats when preservative-free test solution E was used in comparison with Xalatan.

FIG. 2B shows intraocular pressure(IOP) after single topical administration in rats when preservative-free test solution F was used in comparison with Xalatan.

FIG. 2C shows intraocular pressure(IOP) after repeated administrations in rats when preservative-free test solution F was used in comparison with Xalatan.

FIG. 3A shows preliminary stability of latanoprost unit dose eye drop composition during storage at 5° C.

FIG. 3B shows preliminary stability of latanoprost unit dose eye drop composition during storage at 25° C.

### DESCRIPTION OF EMBODIMENTS

The present invention is a method for improving ocular bioavailability of latanoprost from an aqueous eye drop composition by adding an organic amine to the eye drop composition containing latanoprost. The present invention also provides an aqueous eye drop composition comprising latanoprost, wherein the ocular bioavailability of latanoprost is improved by adding an organic amine. Furthermore, the present invention also provides a method for treating ocular hypertension and glaucoma by administering to a subject in need of such treatment an effective amount of an aqueous eye drop composition comprising latanoprost, wherein the ocular bioavailability of latanoprost is improved by adding an organic amine.

This invention enables to provide homogenous and stable, preservative-free aqueous ophthalmic latanoprost solution as a sterile, preferably unit dose type formulation. The aqueous eye drop composition according to the invention is also less irritative to the eye than the currently available commercial product. The aqueous eye drop composition of the present invention also remains stable at room temperature, contrary to the currently available commercial product which requires cold storage (at 2-8° C.).

Within this description, “substantially no preservatives” or “preservative-free” means that the composition(solution) contains absolutely no preservatives, or the composition(solution) contains preservatives at a concentration that is undetectable or does not provide a preservative effect.

“Bioavailability” refers to the degree and rate at which a drug is absorbed and made available in the target tissue. “Ocular bioavailability” refers more specifically to the concentration of a drug in the aqueous humor after topical administration of a drug comprising the aqueous eye drop composition.

“Stability” refers to chemical and physical stability of a drug comprising the aqueous eye drop composition.

The ingredients other than latanoprost and an organic amine may not be particularly limited as far as latanoprost is homogenously and stably dissolved in the aqueous solution. However, it is notable that nonionic surfactants (polysorbates, cremophores etc.) or preservatives are not required in the aqueous eye drop composition of the invention to provide a stable and bioavailable formulation for use in a single dose form.

The concentration of latanoprost in the aqueous eye drop composition and dosing frequency may vary according to the type and condition of the subject to be treated. For example, an aqueous eye drop composition containing latanoprost at a concentration of 0.0005 to 0.01% (w/v), preferably 0.001 to 0.0075% (w/v), more preferably 0.0025 to 0.005% (w/v) may be instilled 1 to 4 times, preferably 1 to 2 times, and more preferably 1 time a day.

As the invention provides improved ocular bioavailability and intraocular pressure(IOP) lowering effect of latanoprost, the amount of latanoprost in the aqueous eye drop composition can be lowered compared to the prior art products, still achieving an effective product.

The organic amine is a water-soluble organic amine, examples of which include organic amines having a hydroxy group, such as monoethanolamine, diethanolamine, triethanolamine, trometamol and meglumine. A preferred organic amine is trometamol.

The concentration of the organic amine is for example in the case of trometamol preferably in the range of from 0.01% to 1% (w/v), more preferably from 0.05 to 0.75% (w/v).

The aqueous eye drop composition according to the invention may also comprise conventional excipients used in ophthalmic compositions, such as buffering agents, solvents, pH adjusters, tonicity agents, viscocity enhancers and the like. Preferred excipients for use in the aqueous eye drop composition of the present invention are viscosity enhancers. Suitable viscosity enhancers include but are not limited to cellulose derivatives, such as hydroxypropyl methylcellulose, sodium carboxymethylcellulose, methylcellulose, polyvinylpyrrolidone, polyvinylalcohols, dextrans and polyacrylic acids. Examples of suitable buffering agents include but are not limited to sodium dihydrogen phosphate dihydrate, boric acid, borax, citric acid, or e-aminocaproic acid. Specific examples of tonicity agents include but are not limited to glycerol, sorbitol, mannitol and other sugar alcohols, propylene glycol, sodium chloride, potassium chloride and calcium chloride

The pH of the aqueous ophthalmic latanoprost solution according to the invention is preferably from 4 to 8, more preferably from 5 to 7. As pH adjusters, common pH adjusting agents such as sodium hydroxide and/or hydrochloric acid may be used.

The aqueous ophthalmic latanoprost solution according to the invention is preferably packaged in a container consisting essentially of polyethylene or in contact with material consisting essentially of polyethylene. The container material may contain minor amounts of other materials than polyethylene, for example polypropylene, polyethylene terephthalate, polyvinyl chloride, acrylic resins, polystyrene, polymethyl methacrylate and nylon 6. The amount of said materials is preferably no more than about 5 to 10% of the total container material.

Containers for packaging and storing the aqueous ophthalmic latanoprost solution according to the invention include all container forms suitable for user-friendly topical ophthalmic delivery, preferably those of unit dose form. Consequently, the containers may be selected for example from the group consisting of bottles, tubes, ampoules, pipettes and fluid dispensers for dispensing minute amounts of sterile fluid.

The containers for the aqueous ophthalmic latanoprost solution according to the invention are preferably manufactured by extrusion blow moulding method. Extrusion blow moulding gives smoother inner surface of the container compared to injection blow moulding, which is commonly used to manufacture for example polyethylene multidose bottles. The smoother inner surface gives better chemical stability of prostaglandins in the polyethylene container compared to polyethylene container manufactured by injection blow moulding. Furthermore, when single-dose containers are used, they are sterilized during the moulding process by heat and no additional sterilization of containers is needed, which also improves stability of prostaglandins in a single-dose container. Conventional techniques for sterilization of prostaglandin eye drop products consist of treatment of the empty plastic bottles with highly toxic ethylene oxide gas before filling-in the solution (see EP 1825855(Santen)) and EP 1349580(Novartis)). The much smoother sterilization process used in the preparation of the eye drop composition of the present invention (heat sterilization during the filling process) is however more recommendable for a unit dose preparation especially in polyethylene containers.

Although unit dose containers are preferred for the purposes of the invention, the aqueous ophthalmic latanoprost solution according to the invention remains soluble, stable and bioavailable also in fluid dispensers for dispensing of minute amounts of germ-free fluid or in any other container-type wherein the aqueous ophthalmic latanoprost solution is in contact with container material consisting essentially of polyethylene. Such fluid dispensers are disclosed for example in U.S. Pat. No. 5,614,172 (Ursapharm).

The preservative-free aqueous ophthalmic latanoprost solution according to the invention can be stored at room temperature in the above mentioned suitable containers, including unit dose pipettes and dispensers. Stability studies have shown that a preservative-free aqueous ophthalmic latanoprost solution according to the invention is stable in a polyethylene container for a long time, at least for 9 months at 5° C. and at 25° C.

In the aqueous eye drop composition comprising latanoprost according to the present invention, it is preferred that the composition comprises 0.0025 to 0.005% (w/v) latanoprost, 0.1 to 1% (w/v) trometamol, optionally buffering agents, pH adjusters, tonicity and viscosity enhancing agents conventionally used in ophthalmic solutions, and substantially no preservatives, in a unit dose container or fluid dispenser consisting essentially of polyethylene or in contact with container material consisting essentially of polyethylene.

The following examples illustrate the invention without limiting the same in any way.

### Example 1

Male albino rabbits of New Zealand White strain were used. In each rabbit, one eye was dosed topically with one of the test solution A, B, C or D and the other eye with the reference solution (Xalatan). The latanoprost-trometamol ratios used in the test solutions A, B, C and D were as follows:

(1) Test Solution A

Latanoprost—Trometamol ratio: 0.013 (Latanoprost concentration 0.005%)

(2) Test Solution B

Latanoprost—Trometamol ratio: 0.017 (Latanoprost concentration 0.005%)

(3) Test Solution C

Latanoprost—Trometamol ratio: 0.017 (Latanoprost concentration 0.005%)

(4) Test Solution D

Latanoprost—Trometamol ratio: 0.029 (Latanoprost concentration 0.0035%)

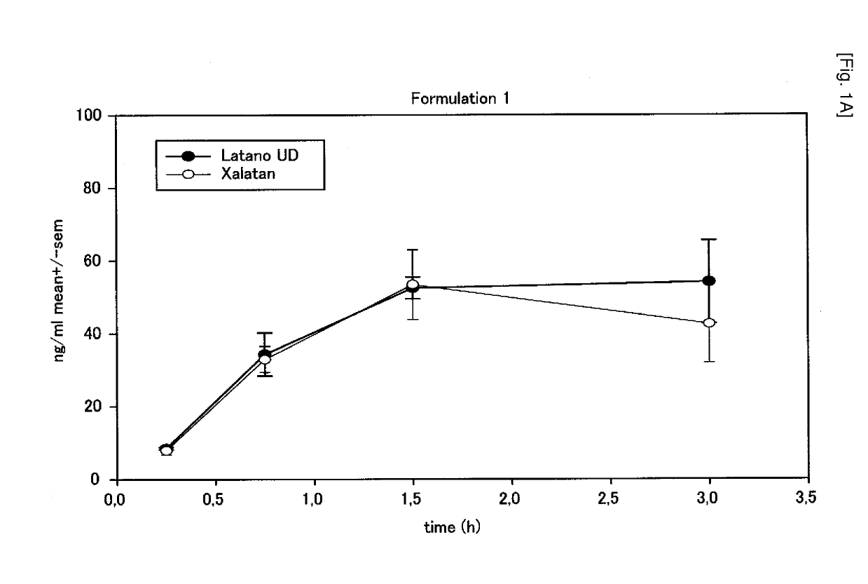

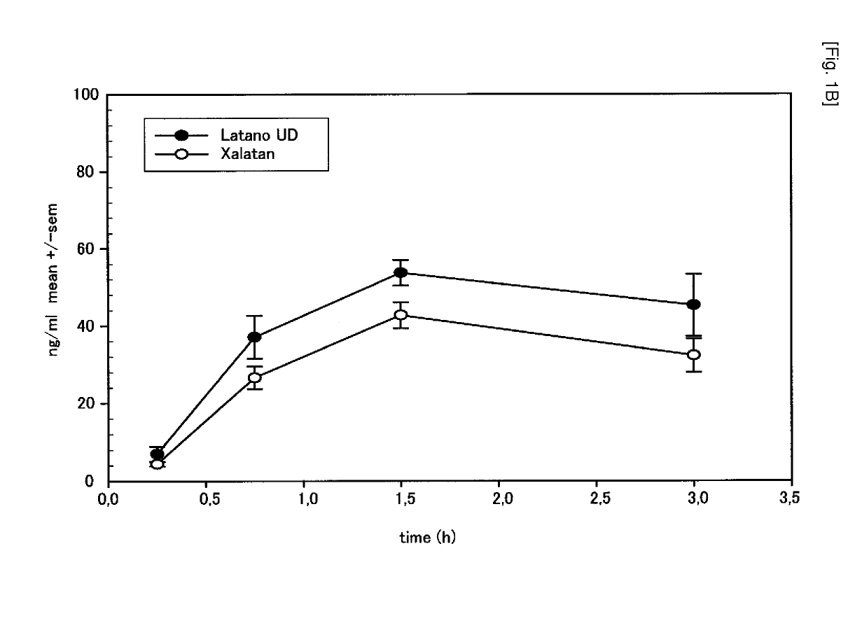

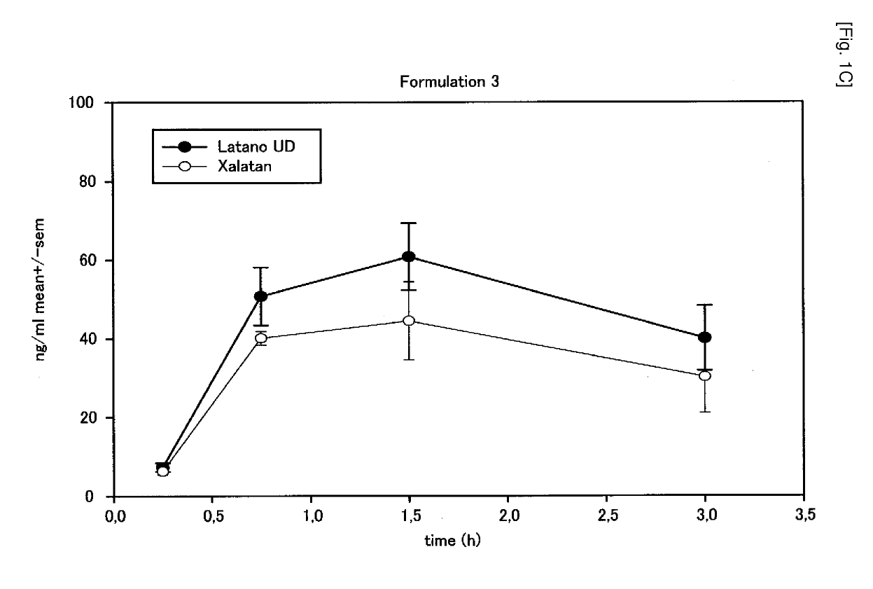

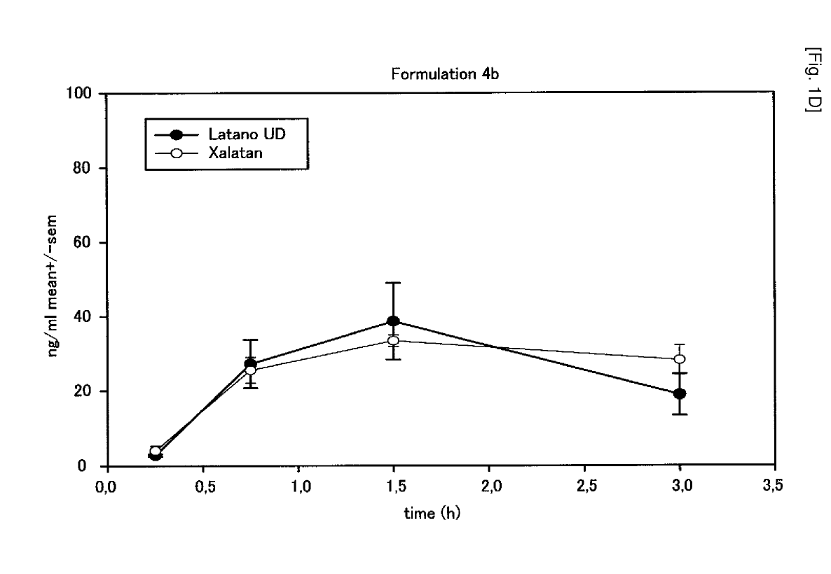

The rabbits were sacrificed at each time point (4 animals per treatment per time point) and aqueous humor sample was taken. The concentration of latanoprost acid was measured using the HPLC/MS method. The results of test solution A, B, C and D are shown in FIGS. 1A, 1B, 1C and 1D, respectively.

FIGS. 1A-1C illustrate absorption of latanoprost from preservative-free test solutions with different latanoprost-trometamol ratio into aqueous humor of rabbit eye in comparison with Xalatan, when latanoprost concentration in the test solution was the same as in Xalatan. In FIG. 1D, latanoprost concentration in the test solution was 70% of the latanoprost concentration in Xalatan.

In FIGS. 1A-1D it can be seen that by increasing the latanoprost-trometamol ratio in preservative-free test solutions transcorneal penetration of latanoprost into aqueous humor of rabbit eye is improved.

### Example 2

Spraque-Dawley rats were used to study the effects of ophthalmic solutions on the TOP. The test solutions E and F were dosed topically (5 ml) either as a single instillation or repeated instillations (for eight days) onto one eye and the reference solution (commercial Xalatan) onto the other. The latanoprost-trometamol ratios used in the test solutions E and F were as follows:

(1) Test Solution E

Latanoprost—Trometamol ratio: 0.020 (latanoprost concentration the same as in Xalatan)

(2) Test Solution F

Latanoprost—Trometamol ratio: 0.029 (latanoprost concentration 70% of Xalatan)

Six to twelve animals were used per study. TOP was measured prior to dosing at 0 h and 2, 4, 6 and 8 hours (or additional 10 hours) after the dosing. Measurement was performed with a TonoLab rebound tonometer.

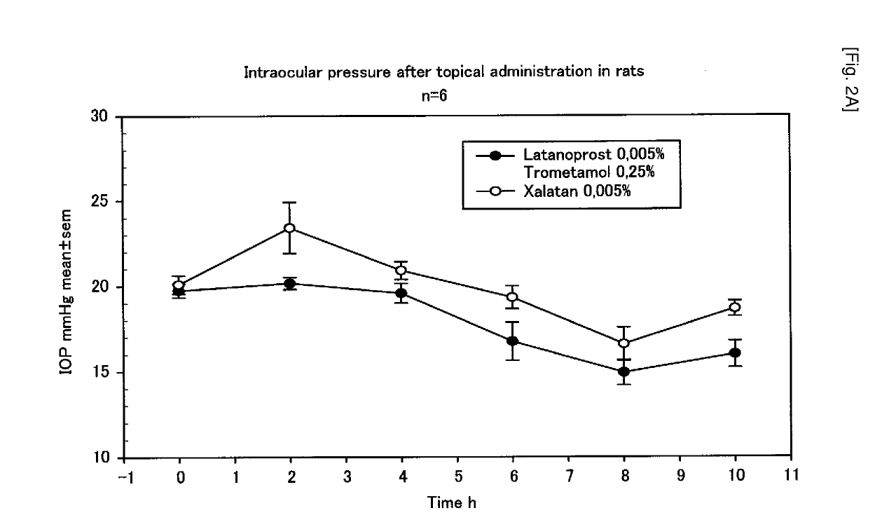

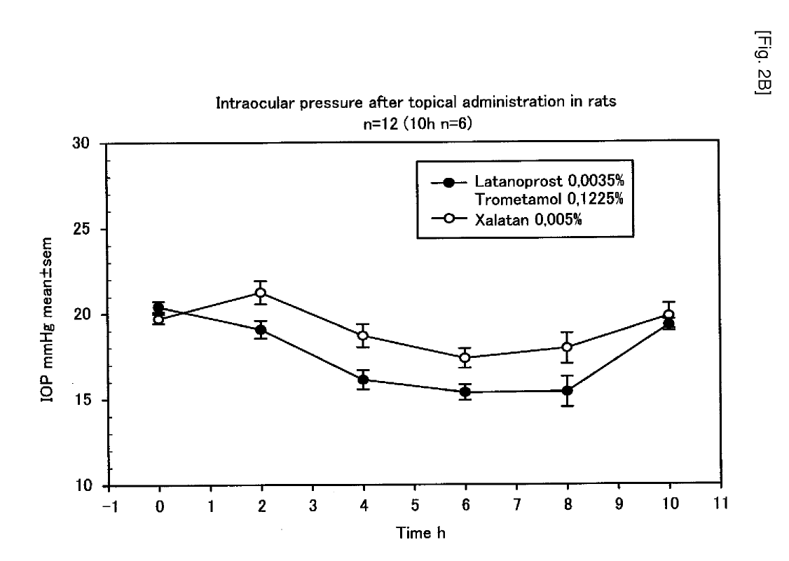

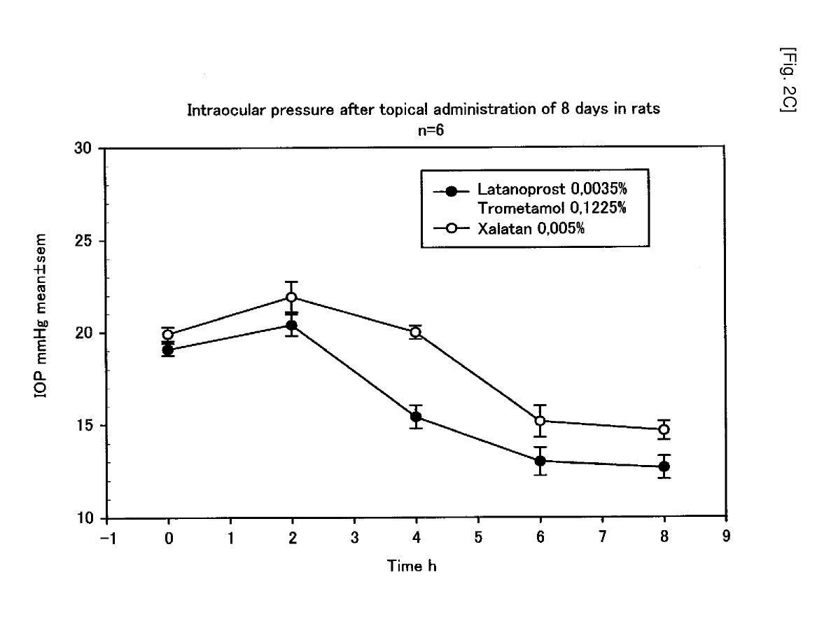

FIGS. 2A-2B show TOP after single topical administration in rats when preservative-free test solutions with different latanoprost-trometamol ratios and different latanoprost concentrations were used in comparison with Xalatan. FIGS. 2B-2C compare the effect on TOP after single administration and repeated administrations (for eight days) of the formulation containing Latanoprost—Trometamol ratio: 0.029 (latanoprost concentration 70% of Xalatan).

From the results it can be seen that the commercial product causes an immediate increase in the TOP and the TOP lowering effect is seen only after several (6 to 8) hours. The compositions according to the invention do not provide a harmful initial increase in the TOP which is common when eye prostaglandins like latanoprost are administered. With the compositions according to the invention the TOP lowering effect starts earlier than with the current commercial product. The TOP lowering effect stays at the same level after repeated instillation as it is after single instillation which loss of efficacy may often be the case with repeated administration of eye prostaglandins like latanoprost. Furthermore, the TOP lowering effect of the composition according to the invention is better than that of the commercial product, even with a latanoprost concentration of only 70% of the amount of latanoprost in the commercial product.

### Example 3

Blinking frequency was studied as a parameter for acute eye irritation of Xalatan and preservative-free latanoprost according to the present invention in albino rabbits of New Zealand White strain. Six rabbits were used per treatment group, and in each rabbit, one eye was dosed topically with one of the test solutions and the other eye served as a non-treated control. Test solutions were instilled 10 times at 30 min intervals during one day. Blinking frequency was measured for one minute after the 1st and 10th instillation. The results are shown in Table 1.

TABLE 1

| | Xal | Xal | Xal | Xal | Xal | Xal | Lat | Lat | Lat | Lat | Lat | Lat |
|---|---|---|---|---|---|---|---|---|---|---|---|---|
| | R/L | R/L | R/L | R/L | R/L | R/L | R/L | R/L | R/L | R/L | R/L | R/L |
| Blinking | 6/5 | 0/0 | 0/0 | 5/4 | 0/0 | 2/0 | 0/0 | 0/0 | 3/2 | 0/0 | 1/0 | 3/3 |
| after the 1^{st} | | | | | | | | | | | | |
| instillation | | | | | | | | | | | | |
| Blinking | 0/3 | 1/3 | 0/4 | 3/6 | 0/3 | 0/3 | 3/4 | 0/1 | 1/1 | 3/2 | 9/11 | 0/0 |
| after the 10^{th} | | | | | | | | | | | | |
| instillation | | | | | | | | | | | | |

Xal = Xalatan group
Lat = Preservative free latanoprost group
R = Right eye (non-treated eye)
L = Left eye (treated eye)

The commercial Xalatan caused a statistically significantly higher blinking frequency compared to non-treated eye after the tenth instillation. The treated eye blinked more frequently than the non-treated eye. In the preservative-free Latanoprost group no significant difference between eyes was detected. In addition, the difference between Xalatan and Latanoprost groups was statistically significant after the tenth instillation when the differences were compared (non-treated eyes were subtracted from the treated eyes p=0.0005, students t-test) blinking frequency being higher in Xalatan group. The compositions according to the invention are better tolerated.

### Example 4

The stability of preservative-free latanoprost in low density polyethylene containers was studied for 9 months at 5° C. and 25° C. The composition of the preservative-free aqueous latanoprost formulation in the study was 0.003% latanoprost, 0.3% trometamol, 0.9% sodium chloride and hydrochloric acid/sodium hydroxide to adjust the pH to 7.0.

0.3 ml of the composition prepared above was filled in the body part of the polyethylene unit dose container and sealed by heating with the upper part of the container. The containers were packaged into paper coated aluminium-polyethylene foil and stored in refrigerator or incubator.

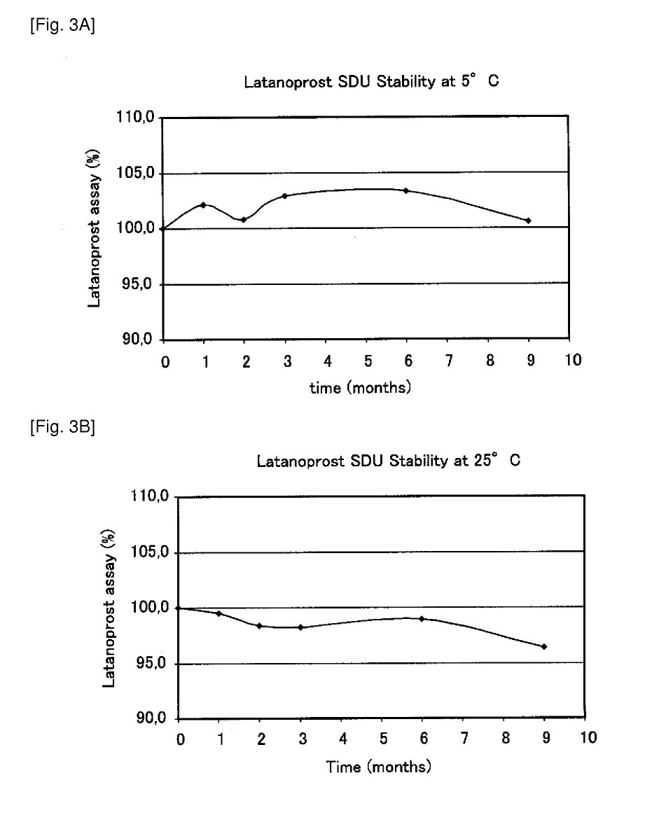

The concentration of latanoprost was measured by HPLC/UV. The results in comparison to time zero value are presented in FIGS. 3A-3B. The results show that latanoprost concentration remains at the same level at 5° C. and 25° C. at least for nine (9) months.

1. A method for improving ocular bioavailability of latanoprost from an aqueous eye drop composition by adding an organic amine to the eye drop composition containing latanoprost.

2. The method according to claim 1, wherein the organic amine is an organic amine having a hydroxy group.

3. The method according to claim 2, wherein the organic amine having a hydroxy group is trometamol.

4. The method according to claim 1, wherein the aqueous eye drop composition contains substantially no preservatives.

5. The method according to claim 1, wherein the aqueous eye drop composition contains viscosity enhancing agents.

6. An aqueous eye drop composition comprising latanoprost, wherein the ocular bioavailability of latanoprost is improved by adding an organic amine.

7. The aqueous eye drop composition according to claim 6 wherein the organic amine is an organic amine having a hydroxy group.

8. The aqueous eye drop composition according to claim 7, wherein the organic amine having a hydroxy group is trometamol.

9. The aqueous eye drop composition according to claim 7 which contains substantially no preservatives or non-ionic surfactants.

10. The aqueous eye drop composition according to claim 6 which further comprises viscosity enhancing agents.

11. The aqueous eye drop composition according to claim 6, which comprises 0.0025 to 0.005% (w/v) latanoprost, 0.1 to 1% (w/v) trometamol, optionally buffering agents, pH adjusters, tonicity and viscosity enhancing agents conventionally used in ophthalmic solutions, and substantially no preservatives, in a unit dose container or fluid dispenser consisting essentially of polyethylene or in contact with container material consisting essentially of polyethylene.

12. A method for treating ocular hypertension and glaucoma by administering to a subject in need of such treatment an effective amount of an aqueous eye drop composition comprising latanoprost, wherein the ocular bioavailability of latanoprost is improved by adding an organic amine.
